(19) United States
(12) Patent Application Publication
(10) Pub. No.: US 2007/0142829 A1
(43) Pub. Date: Jun. 21, 2007

(54) In-vivo interstitial antennas

(75) Inventors: Hee-Ran Ahn, Gyeongsangbuk-do (KR); Bumman Kim, Gyeongsangbuk-do (KR); Kwyro Lee, Seoul (KR)

Correspondence Address: FOLEY AND LARDNER LLP;SUITE 500, 3000 K STREET NW, WASHINGTON, DC, 20007, US

(73) Assignee: Pohang University of Science and Technology

(21) Appl. No.: 11/311,152

(22) Filed: Dec. 20, 2005

(51) Int. Cl. A61B 18/04; A61N 1/00

(52) U.S. Cl. 606/33; 607/156

(57) ABSTRACT

Disclosed are in-vivo interstitial antennas (IVIAs) for thermal treatment and deactivation of tumors by means of microwaves. An IVIA comprises a microwave monopole antenna (MMA) and a medical catheter, and the MMA is inserted into the medical catheter to form the IVIA. The MMA comprises coaxial cable and three types of capacitors. The coaxial cable consists of first and second conductors and a first insulator, and only the first conductor extends less than a quarter wavelength. The first capacitor is located around the end of the extended first conductor and includes the second insulator and the third conductor. The second and third capacitors are located between the first capacitor and the apertures of the MMAs and have about same function. Because of arbitrarily changed input impedance of the first capacitor, almost perfect matching can be achieved and desirable temperature distributions can be obtained due to the second and third capacitors.

### BACKGROUND OF THE INVENTION

1. Field of the Invention

The present invention relates to in-vivo interstitial antennas (IVIAs). More precisely, the invention relates to IVIAs for thermal treatment and deactivation of deep-seated tumors including cancers in a human body by means of microwave.

2. Background of the Related Art

Conventional surgical operations have been performed for tumors including cancers in a human body. However, such operations not only result in considerable cost and recovery time, but also expose the patients to high risk of secondary infection. To overcome the problems mentioned above, the IVIA using the microwave can be used to treat and deactivate tumors including cancers without any surgical operation. Mainly due to less expense, easy operation and short recover time, the use of the IVIAs has recently been on a dramatic increase, and many studies have been published. Details on a conventional typical IVIA will be described.

FIG. 1(a) illustrates cross sectional view of the coaxial cable which is main material of the IVIAs, and how the conventional typical IVIA operates in a human body is described in FIG. 1(b)

The coaxial cable 120 comprises a first conductor 110, a first insulator 111, and a second conductor 112 as shown in FIGS. 1(a) and (b). When the first conductor 110 extends approximately λ_{g}/4 (λ_{g}: wavelength in the medium), a microwave monopole antenna (MMA) is formed and inserted into a medical catheter 114 to avoid direct contact between the MMA and human body tissues. Therefore, the IVIA consists of the MMA and plastic medical catheter and the medical catheter 114 is a harmless plastic tube with a dielectric constant. For easy fabrication of the IVIAs in this invention, air fills the gap 113 between the MMA and plastic catheter, whereas saline has been used for the gap of conventional IVIAs. FIG. 1(b) describes a situation where a conventional typical IVIA is inserted into an assumed human body organ and epidermal tissues 115 are shown around the IVIA.

When current is applied to the IVIA through the first conductor 110 of the coaxial cable 120 in FIG. 1(b), positive charges are produced around the IVIA and electric fields are, therefore, generated between the positive charges and the distant negative charges. Since the human body tissues is conducting and lossy media, heat is generated by the electric fields and temperature rises around the targeting heating area where the IVIA is inserted in the human body. The temperatures of more than 43 degrees centigrade can be used for treatment and deactivation of tumors including cancers in a human body.

FIG. 2 shows conventional MMAs. The conventional MMA in FIG. 2(a) is the most common one whose, first conductor 110 is extended. That in FIG. 2(b) has the third conductor 210 in a quadrilateral form which is wider than diameter of the first conductor. Therefore, more current can be concentrated around the third conductor 210. For that in FIG. 2(c), an end is located around the extended first conductor and includes a third conductor 212 in the form of a metal tube concentrically surrounding the first conductor 110 with both ends open, and the second insulator 212 fills the gap between the first 110 and third 212 conductors.

When IVIA is used for a human body, the IVIA matching is most important factor to be considered. If the IVIA is not matched, the thermal energy can not be concentrated around the targeting heating area, and the microwave source may be destroyed by unavoidable reflected power. In addition, thermal pattern should also be taken into consideration and isothermal line contour with 43 degrees centigrade is desired to be similar to the shape of tumors including cancers to protect healthy surrounding tissues during the microwave treatment.

However, conventional IVIAs have been poorly matched due to perfect matching methods unavailable. The poor matching results in poor thermal energy concentration and undesired thermal pattern, and damage to healthy surrounding tissues can therefore occur.

### SUMMARY OF THE INVENTION

An objective of the present invention is to provide IVIAs in order to have good matching, desired thermal pattern, high thermal efficiency and little damage to healthy surrounding tissues.

In addition, it is another object of the present invention to provide the IVIAs for optimizing the thermal pattern to treat and deactivate tumors including cancers in a human body.

To accomplish the above objects, according to one aspect of the invention, an IVIA using microwaves is provided for thermal treatment of tumors including cancers in a human body. The IVIA consists of a MMA and a medical catheter in the form of dielectric tube with a dielectric constant, and the MMA is inserted into the medical catheter to form the IVIA. The MMA consists of the coaxial cable with the first conductor extending and a first capacitor located around the end of the extended first conductor. The coaxial cable and the first capacitor will be explained in more details.

Coaxial cable, main material of the MMA, includes a first conductor having a cylindrical form and being used for applying current; a second conductor in the form of a metal tube concentrically surrounding the first conductor and used for ground when applying the current; a first insulator having a dielectric constant and filling the gap between the first and second conductors to insulate from each other; and only the first conductor extending less than a quarter wavelength.

A first capacitor is located around end of the extended first conductor, has very small length compared to a quarter wavelength and includes a third conductor in the form of a metal tube concentrically surrounding the extended first conductor with one end at the end of the first conductor closed and connected with the first conductor while the other end being open; and a second insulator having a dielectric constant and filling the gap between the first and third conductors.

According to an embodiment of the invention, the IVIA includes the second insulator of the first capacitor, by which opposite charges can be induced on the side-surface of the third conductor when current flows through the first capacitor. Input impedance of the IVIA can be arbitrarily changed in accordance with the length of the first capacitor and perfect IVIA matching can therefore be possible.

According to another aspect of the invention, an IVIA using microwaves is given to treat and deactivate tumors including cancers in a human body. The IVIA consists of a MMA and a medical catheter in the form of dielectric tube with a dielectric constant, and the MMA is inserted into the medical catheter to form the IVIA. The MMA comprises coaxial cable with only the first conductor extending, a first and second capacitors, which will be explained in more details.

Coaxial cable, main material of the MMA, includes a first conductor having a cylindrical form and being used for applying current; a second conductor in the form of a metal tube concentrically surrounding the first conductor and used for ground when applying the current; and a first insulator having a dielectric constant and filling the gap between the first and second conductors to insulate from each other; and only the first conductor extending less than a quarter wavelength.

The first capacitor is located around the extended first conductor, having a certain length and including a third conductor in the form of a metal tube concentrically surrounding the extended first conductor with one end at the end of the first conductor closed and connected with the first conductor while the other end being open; and a second insulator filling the gap between the first and third conductors.

The second capacitor is located between the first capacitor and the MMA aperture where the first conductor starts to extend, has a certain length and includes a fourth conductor in the form of metal tube concentrically surrounding the extended first conductor with both ends open and a third insulator filling the gap between the first and fourth conductors.

According to an embodiment of the invention, due to the first capacitor, an IVIA can be perfectly matched. In addition, an IVIA with desirable thermal pattern can also be provided due to the second capacitor. The extended first conductor is common with coaxial cable, the first and second capacitors.

According to another aspect of the invention, an IVIA using microwaves is supplied for thermal treatment and deactivation of tumors including cancers in a human body. The IVIA consists of a MMA and a medical catheter in the form of dielectric tube with a dielectric constant, and the MMA is inserted into the medical catheter to form the IVIA. The MMA consists of coaxial cable with the first conductor extending, a first, second and third capacitors, which will be described in more detail.

Coaxial cable, main material of the MMA, includes a first conductor having a cylindrical form and being used for applying current; a second conductor in the form of a metal tube concentrically surrounding the first conductor and used for ground when applying the current; a first insulator having a dielectric constant and filling the gap between the first and second conductors to insulate from each other; and only the first conductor extending less than a quarter wavelength.

The first capacitor with a certain length is located around end of the extended first conductor and includes a third conductor in the form of a metal tube concentrically surrounding, the extended first conductor with one end at the end of the first conductor closed and connected with the first conductor while the other end being open; and a second insulator filling the gap between the first and third conductors.

The second capacitor with a certain length is located between the open end of the first capacitor and the MMA aperture and includes a fourth conductor in the form of a metal tube concentrically surrounding the first conductor with both ends being open, and a third insulator filling the gap between the first and fourth conductors.

The third capacitor with a certain length is located between the second capacitor and the MMA aperture and includes a fifth conductor in the form of a metal tube concentrically surrounding the first conductor with both ends open, and a fourth insulator filling the gap between the first and fifth conductors.

According to an embodiment of the invention, the second and third capacitors include the third and fourth insulators, each having arbitrary dielectric constants. The first conductor is common with the first, second and third capacitors.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1a shows a cross sectional view of the coaxial cable used for the IVIAs;

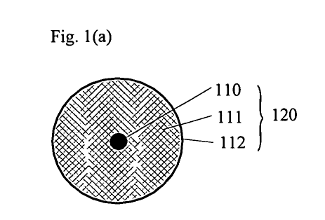

FIG. 1b illustrates how a typical IVIA operates inserted in an assumed human organ;

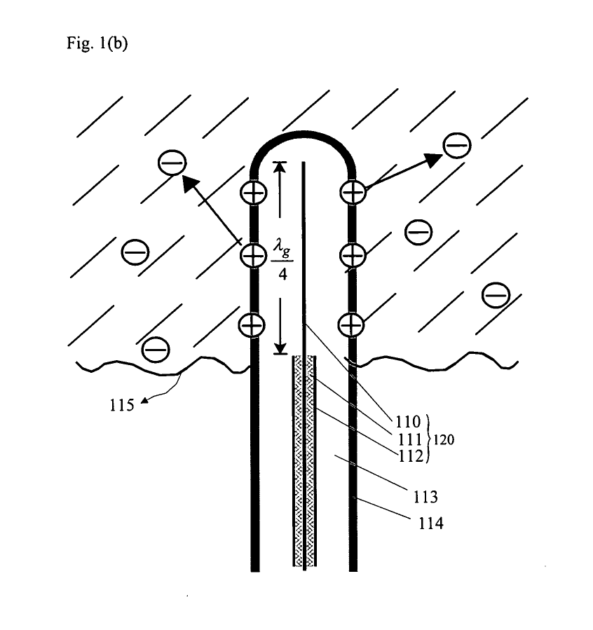

FIG. 2 shows conventional representative MMAs;

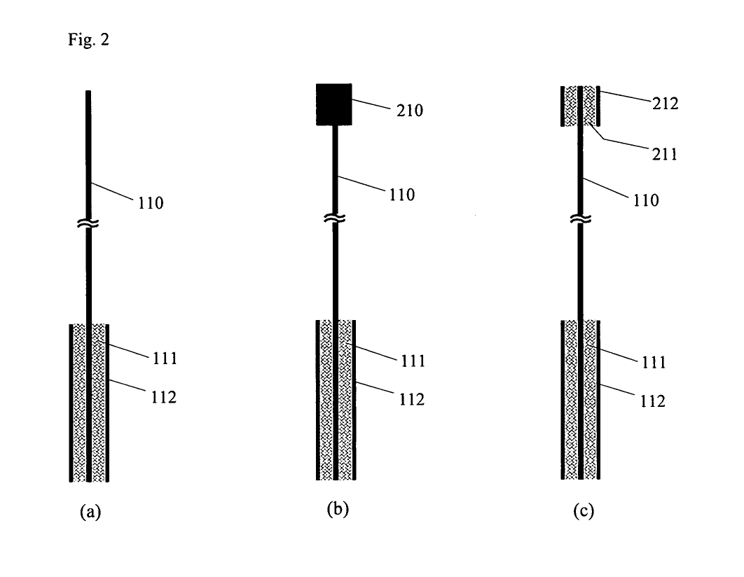

FIG. 3 describes an IVIA according to the first embodiment of the invention;

FIG. 4 shows the first capacitor of the IVIAs;

FIG. 5 compares the first embodiment of the invention with the conventional IVIAs, in terms of electric energy density, which is proportional to temperature;

FIG. 6 shows compared measured and calculated matching performances of the IVIA, according to the first embodiment of the invention;

FIG. 7(a) shows a schematic diagram of the second embodiment of the invention;

FIG. 7(b) shows measured matching performance of the second embodiment of the invention is compared with calculated one;

FIG. 8(a) shows a schematic diagram of the third embodiment of the invention;

FIG. 8(b) shows Measured matching performance of the third embodiment of the invention is compared with calculated one;

FIG. 9(a) shows compared temperature distributions of the IVIAs according to the first and second embodiments of the invention;

FIG. 9(b) shows compared temperature distributions of the IVIAs according to the first and third embodiments of the invention; and

FIG. 9(c) shows compared temperature distributions of the IVIAs according to the second and third embodiments of the invention.

### DETAILED DESCRIPTION OF THE PREFERRED EMBODIMENT

The preferred embodiments of the invention will be hereafter described in detail, with reference to the accompanying drawings.

### First Embodiment

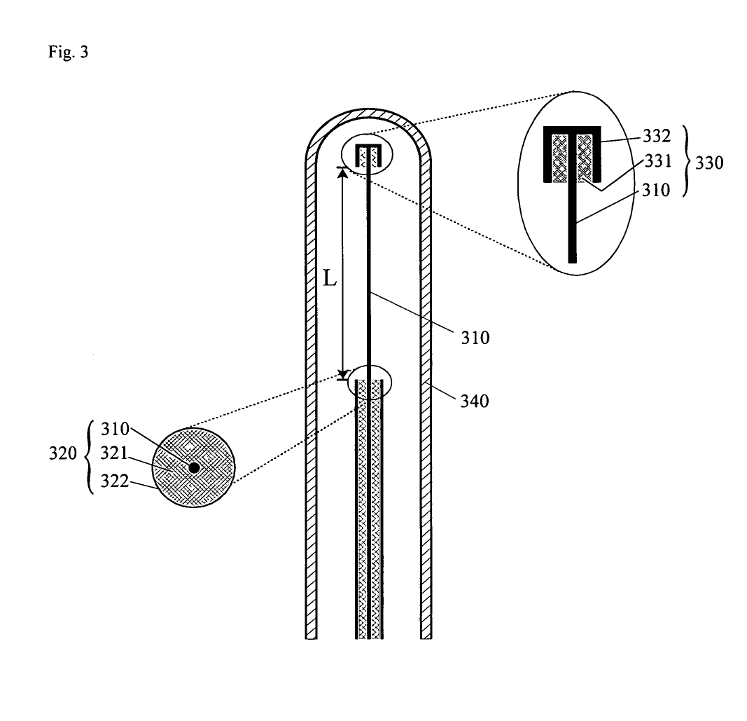

FIG. 3 shows a schematic view of an IVIA according to a first embodiment of the invention. As illustrated in FIG. 3, the IVIA comprises a coaxial cable 320 consisting of a first conductor 310, a first insulator 321 and a second conductor 322, a first capacitor 330 and a catheter 340.

Only the first conductor 310 extends less than a quarter wavelength, or, slightly longer than L and is used for applying current.

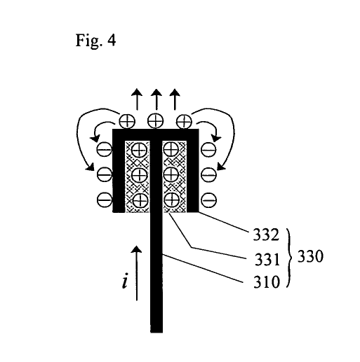

The first capacitor 330 is located around the end of the extended first conductor 310 and includes a third conductor 332 in the form of a metal tube concentrically surrounding the extended first conductor with one end at the of the first conductor closed and connected with the first conductor while the other end being open; and a second insulator 331 having a dielectric constant and filling the gap between the first and third conductors. Details on the above description will be explained in FIG. 4.

The coaxial cable 320, main material of the IVIA, comprises the first conductor 310, the first insulator 321 and a second conductor 322. In addition, the first capacitor 330 is located around the end of the extended first conductor.

Here, the first insulator 321 of the coaxial cable and the second insulator 331 of the first capacitor can be arbitrary but the same insulators are used for convenient fabrication.

The coaxial cable 320 with the first conductor extending slightly longer than L and the first capacitor 330 are composed of a MMA, all of which are inserted into the medical catheter 340 in the form of a dielectric tube with a dielectric constant to form the IVIA. The gap between the MMA and the catheter 340 comprises air.

FIG. 4 shows a schematic diagram of the first capacitor of the IVIA which is very important for the invention. The first capacitor 330 is located around the end of the extended first conductor 310, has a certain length and includes a third conductor 332 in the form of a metal tube concentrically surrounding the extended first conductor 310 with one end at the end of the first conductor 310 closed and connected with the first conductor 310 while the other end open; and the second insulator 331 having a certain dielectric constant and filling the gap between the first 310 and third 332 conductors.

In addition, the shape of the closed end of the third conductor of the first capacitor 330 may be flat or convex. In such a case, the cross sectional area of the third conductor 332 is much larger than that of the first conductor.

Hereafter, details on operation of the IVIA according to the first embodiment of the invention will be described.

When current is applied to the first conductor 310 of the coaxial cable 320, the current is, along the first conductor 310, transmitted to the first capacitor 330 located around the end of the extended first conductor 310. During the current flows through the first capacitor, opposite charges are induced by the second insulator 331 and accumulated on the side-surface of the third conductor 332. That is, according to the first embodiment of the invention, the IVIA has positive charges during the current flow as shown in FIG. 4. In addition, negative charges are, due to the second insulator 331, induced on the side-surface of the third conductor 332 of the first capacitor 330

Due to larger cross sectional area of the third conductor 332 than that of the first conductor 310, the current spreads faster on the surface of the closed end of the first capacitor 330 when it arrives at the end of the first conductor 310. Negative charges induced on the side-surface of the third conductor 332 make the current on the closed end of the third conductor 332 flow down and stay around the open end of the third conductor 332. Because of the induced negative charges on the side-surface, the positive charges on the closed end of the third conductor 332 do not stay and flow down. Therefore, the electric field intensity on the end of IVIA becomes weak and healthy surrounding tissues can be protected.

Given that the surface area of the third conductor 332 is substantially larger than that of the conventional antenna, more current can be concentrated, which results in better thermal efficiency and current concentration.

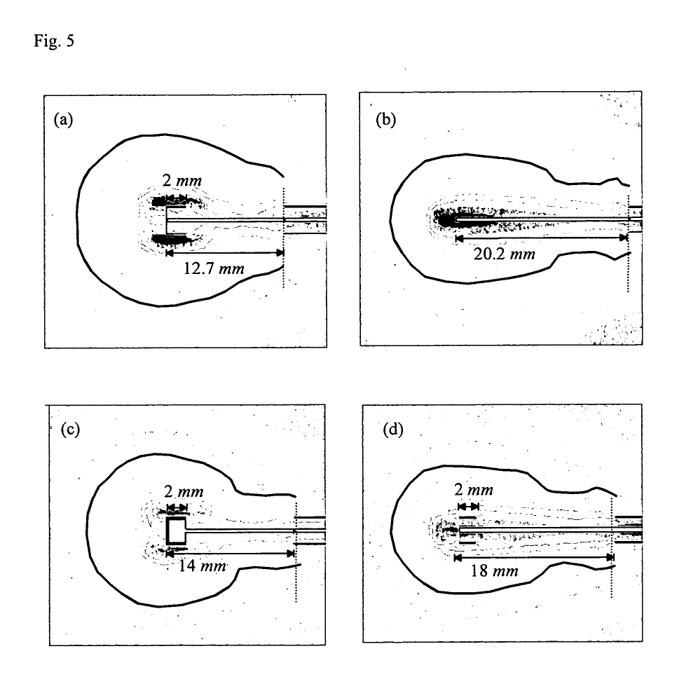

FIG. 5 shows electric energy densities of the IVIAs where the IVIA according to the first embodiment of the invention is in FIG. 5(a), while those of the conventional IVIAs are in FIGS. 5(b)-(d). The electric energy density is proportional to temperature and the temperature decreases with the distance from the IVIAs. Dimensions of the IVIAs are given in Table 1. For the simulations, air fills the gaps between the MMAs and medical catheters and is also used as ambient mediums. The electric energy densities are compared based on simulation results by a 3D-electro magnetic field simulator, computer simulation technology (CST) Microwave Studio, version 4.2.

As illustrated in FIG. 5, when the IVIAs are heated by microwaves, thermal efficiency can be determined by the temperatures around IVIAs. The solid lines indicate the same electric energy densities.

Since the shape of tumors including cancers in a human body is, in general, oval, the desirable temperature isothermal line should be oval like the solid line generated by the IVIA in FIG. 5(a) according to the first embodiment of the invention. The solid line area in FIG. 5(a) is larger than other ones in FIGS. 5(b), (c) and (d). So, the thermal efficiency of the IVIA according to the first embodiment of the invention is better than any other one in FIGS. 5(b), (c) and (d). Nevertheless, the length of the IVIA in 5(a) is the shortest among those of the conventional IVIAs in FIG. 5(b)-(d). The distinctive structure of the first capacitor gives excellent performances of the shortest length and oval type of thermal distribution of the IVIA according to the first embodiment of the invention.

TABLE 1

| Coaxial cable | Radius of first conductor | 0.29 mm |
|---|---|---|
| | Radius of second conductor | 1.4 mm |
| | Dielectric constant | 2.1 |
| Catheter | Inner diameter | 2.3 mm |
| | Outer diameter | 4.2 mm |
| | Dielectric constant | 5.1 |

| | |
|---|---|
| Ambient media | air |

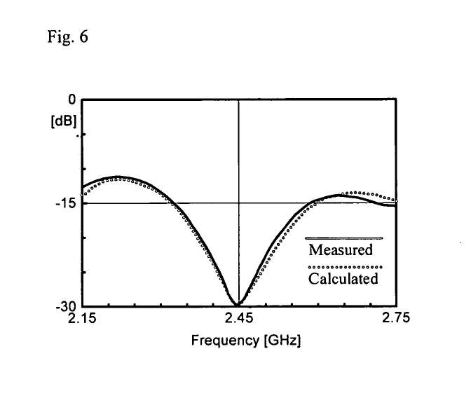

Measured and calculated matching performances of the IVIA according to the first embodiment of the invention are compared in FIG. 6. Fabrication data are listed in Table 2. For the measurements, a vector analyzer is used and power is fed into the first conductor of the IVIA immersed in muscle phantom whose dimensions are 10 cm×10 cm×10 cm. The solid line are the measured results, while the dotted one the calculated ones. The measured matching performance at 2.45 GHz is −28.377 dB, which is the best recorded.

TABLE 2

| | | |
|---|---|---|
| Coaxial cable | Radius of first conductor | 0.145 mm |
| | Radius of second conductor | 0.7 mm |
| | Dielectric constant | 2.1 |
| Catheter | Inner diameter | 1.15 mm |
| | Outer diameter | 2.1 mm |
| | Dielectric constant | 5.1 |
| Human tissues | Dimension | 10 cm × 10 cm × 10 cm |
| (Muscle) | Dielectric constant | 52.7 + j 13.3 |

### Second Embodiment

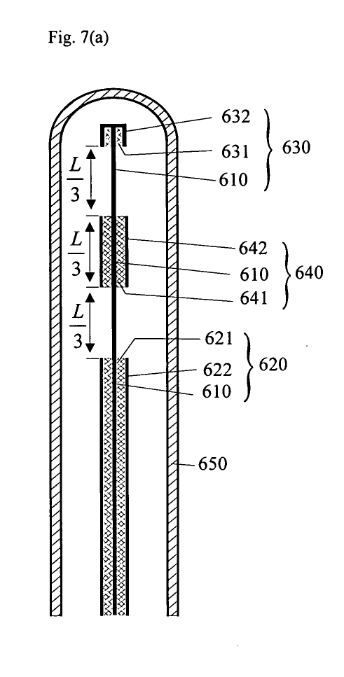

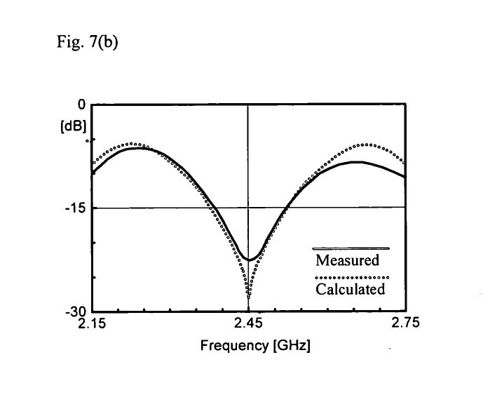

FIG. 7(a) describes an IVIA according to the second embodiment of the invention. As illustrated in FIG. 7(a), the IVIA according to the invention includes coaxial cable 620 with the first conductor 610 extending less than a quarter wavelength, or slightly longer than L, a first capacitor 630, a second capacitor 640 and a catheter 650. FIG. 7(b) compares the measured matching performance of the IVIA according to the second embodiment of the invention with the calculated one, indicating the measured matching result at 2.45 GHz is −21.9 dB.

The first conductor 610 is a conducting material and used as the central axis of the IVIA.

The coaxial cable 620 comprises a first conductor 610 having a cylindrical form and used for applying current; a second conductor 622 in the form of metal tube concentrically surrounding the first conductor and used for ground when current applied; a first insulator 621 having a dielectric constant and filling the gap between the first and second conductors to insulate from each other; and the only first conductor extending slightly longer than L.

The first capacitor 630 with a certain length is located around end of the extended first conductor 610 and includes a third conductor 632 in the form of a metal tube concentrically surrounding the extended first conductor with one end at the extended first conductor closed and connected with the first conductor while the other end being open; and a second insulator 631 having a dielectric constant and filling the gap between the first conductor 610 and the third conductor 632. The first conductor is common with the first capacitor and the coaxial cable.

The second capacitor 640 is located in the middle between the open end of the first capacitor 630 and the MMA aperture and comprises a fourth conductor 642 in the form of a metal tube concentrically surrounding the first conductor with both ends open; and a third insulator 641 having a dielectric constant and filling the gap between the first conductor 610 and the fourth conductor 642.

According to the second embodiment of the invention, assuming the distance between the open end of the first capacitor 630 and the MMA aperture is L, the second capacitor 640 is L/3 long. A distance between the first capacitor 630 and the second capacitor 640, a distance between the second capacitor 640 and the coaxial cable 620, and a length of the second capacitor 640 are the same. And, a space surrounding the coaxial cable 620, the first capacitor 630, and the second capacitor 640 comprises air.

In addition, the first 621, second 631 and third 641 insulators are the same for easy fabrication.

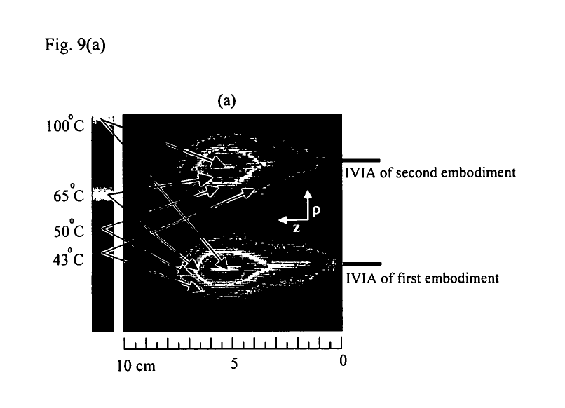

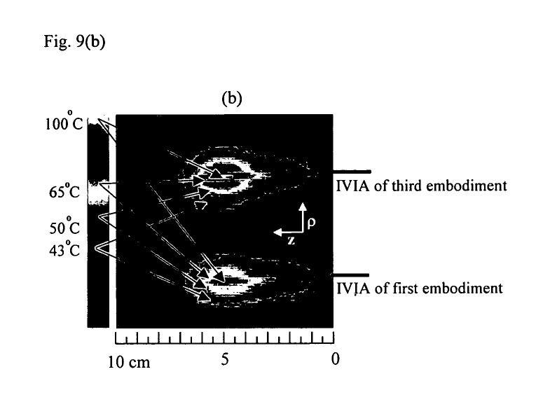

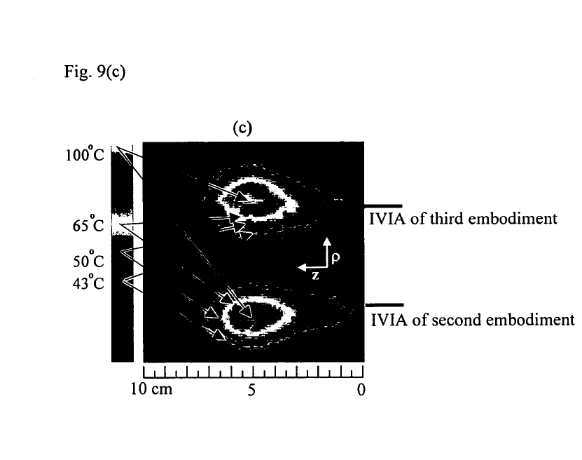

When power is fed into the first conductor 610, opposite charges are accumulated on surface of the fourth conductor 642. Electric fields are generated between the opposite charges on the surface of the fourth conductor 642 and charges staying around the open end of the third conductor 632. Between the opposite charges on the surface of the fourth conductor 642 and charges accumulated around the MMA aperture, electric field also produced. Therefore, the second capacitor 640 is similar to a kind of electric bridge connecting the coaxial cable 620 with the first capacitor 630 when current is flowing. Therefore, the second capacitor 640 optimizes the temperature distributions more similar to the shape of tumors including cancers in a human body. Details on the second capacitor 640 will be explained in following FIG. 9.

The MMA consists of the coaxial cable 620 with the first conductor 610 extended, the first 630 and second 640 capacitors, all of which are inserted into a medical catheter to form the IVIA. Air fills the gap between the MMA and catheter.

According to the second embodiment of the invention, same as the first, one end of the first capacitor 630 is closed, more precisely; one end of the third conductor 632 at the end of the first conductor 610 is closed and connected with the first conductor.

In addition, the closed end of the first capacitor 630 may be flat or convex. In such a case, since the cross sectional area of the closed end is much larger than that of the first conductor 610, current reaching the end of the extended first conductor 610 spreads faster on the closed surface of the third conductor 632.

According to the second embodiment of the invention, the IVIA includes the second 631 and third 641 insulators. Due to the insulators, opposite charges are, during the current flows through the first and, second capacitors, induced and accumulated on the side surface of the third conductor 632 and surface of the fourth conductor 642.

### Third Embodiment

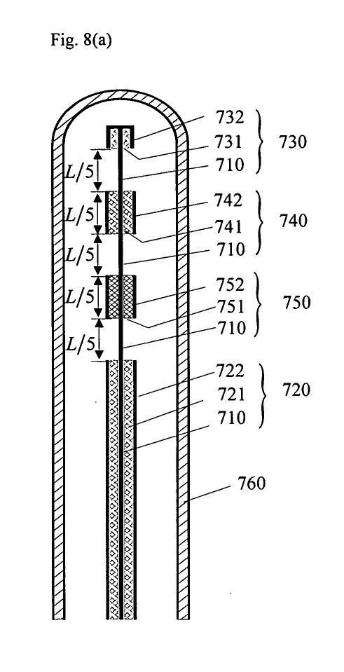

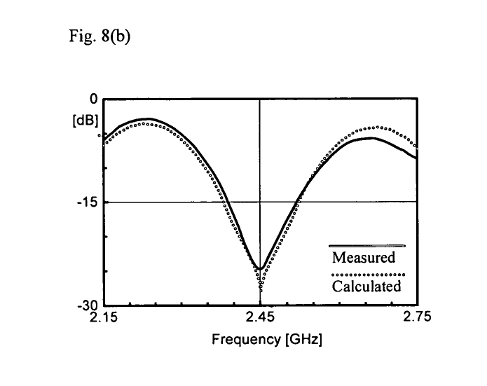

FIG. 8(a) shows a schematic diagram of an IVIA according to the third embodiment of the invention. The IVIA includes a coaxial cable 720 with a first conductor 710 extending less than a quarter wavelength, or, slightly longer than L, a first capacitor 730, a second capacitor 740, a third capacitor 750 and a catheter 760. FIG. 8(b) compares the measured matching performance of the IVIA according to the third embodiment of the invention with the calculated one, indicating the measured matching result is −24.4 dB at 2.45 GHz.

The first conductor 710 is common with the coaxial cable, the first, second and third capacitors and used as the central axis of the IVIA.

The coaxial cable 720 comprises a first conductor 710 having a cylindrical form and used for applying current; a second conductor 742 in the form of a metal tube concentrically surrounding the first conductor and used for ground when current is applied; and a first insulator 721 having a dielectric constant and filling the gap between the first 710 and second 722 conductors to insulate from each other, and only the first conductor extending less than a quarter wavelength, or slightly longer than L.

The first capacitor 730 with a very small length is located around end of the extended first conductor 710 and includes a third conductor 732 in the form of a metal tube concentrically surrounding the extended first conductor with one end closed and connected with the first conductor while the other end being open; and a second insulator 731 having a certain dielectric constant and filling the gap between the first 710 and third 732 conductors.

The second 740 and third 750 capacitors are located between the first capacitor 730 and the MMA aperture, and the functions of the two capacitors are about same. The second capacitor 740 comprises a fourth conductor 742 in the form of a metal tube concentrically surrounding the extended first conductor with both ends open; and a third insulator 741 filling the gap between the first 710 and the fourth 742 conductors.

The third capacitor 750 is located between the second capacitor 740 and the MMA aperture and comprises a fifth conductor 752 in the form of a metal tube concentrically surrounding the first conductor with both ends open; and a fourth insulator 751 having a certain dielectric constant and filling the gap between the first 710 and the fifth 752 conductors.

In such a case, the second 740 and third 750 capacitors are of the same length of L/5. The second capacitor 740 begins at L/5 from the open end of the first capacitor and the third capacitor 750 begins at L/5 from the one end of the second capacitor 740. A distance between the first and the second capacitors, a distance between the second capacitor 740 and the third capacitor 750, a distance between the third capacitor 750 and the coaxial cable 720, and a length of the second and the third capacitors 740,750 are the same.

In addition, the first 721, second 731, third 741 and fourth 751 insulators are same with each other for easy fabrication in this invention.

The MMA comprises the coaxial cable 720 with the first conductor extending less than a quarter wavelength, or, slightly longer than L, the first capacitor 730, the second capacitor 740 and the third capacitor 750, all of which are inserted into a medical catheter 760 in the form of a dielectric tube having a dielectric constant to form the IVIA according to third embodiment of the invention. The gap between the catheter 760 and the MMA comprises air.

According to the third embodiment of the invention, one end of the first capacitor 730, more precisely; one end of the third conductor 732 is closed and connected with the extended first conductor 710, while the other end is open.

In addition, the closed end of the first capacitor 730 may be flat or convex. In such a case, since the cross sectional area of the third conductor 732 is much larger than that of the first conductor 710, current reaching the end of the first conductor 710 spreads faster on the surface of the closed end of the third conductor 732 than along the first conductor 710.

According to the third embodiment of the invention, opposite charges can be induced on the surface of the third 732, fourth 742 and fifth 752 conductors due to the second 731, third 741 and fourth 751 insulators during the current flows through the first 730, second 740, and third 750 capacitors. The induced opposite charges contribute to the desirable temperature distribution pattern of the IVIA for the treatment and deactivation of tumors including cancers by means of microwaves.

When designing the IVIAs of the present invention, matching and temperature distributions should be considered. Using the first capacitor 730, the IVIA according to the third embodiment of the invention can be perfectly matched like the first and second embodiments of the invention. Using the second 740 and third 750 capacitors, desirable temperature distribution can be obtained like the second embodiment of the invention. Each embodiment of the invention will be compared with each other and the compared results will be plotted in FIG. 9.

Temperature distribution is one of important factors to be considered, because any temperature of more than 43 degrees centigrade can be used for the treatment and deactivation of tumors including cancers in a human body. The temperature distributions of the IVIAs according to the first to third embodiments of the invention are pictured by a IRCON (Inspect IR 500 PS) digital camera and they are compared with each other in FIG. 9.

Temperature measurements are carried out in the following ways. Two IVIAs are inserted into a 10 cm×10 cm×10 cm muscle phantom and then microwave power is fed into each IVIA. The distance between two IVIAs is 5 cm and four thermometer fiber optic sensors are attached at four different points on the IVIAs. If any of four reaches at 100 degrees centigrade, the microwave power supplied by a generator is automatically stopped and half of the phantom should be separated as soon as possible to take pictures.

If the shape of 43 degrees centigrade isothermal line is more similar to egg, the IVIA is better for the treatment and deactivation of tumors including cancers because of the inherent shape of the tumors including cancers.

FIG. 9(a) shows temperature distributions patterns of the IVIAs according to the first and second embodiments of the invention. Here, the Z direction is the longitudinal axis of the IVIA and the ρ direction is perpendicular to the Z direction. As illustrated in FIG. 9(a), the isothermal contour with 43 degrees centigrade of the second embodiment of the invention is shorter in terms of the Z direction and longer in terms of the ρ direction than that of the first embodiment of the invention.

FIG. 9(b) illustrates the temperature distribution patterns of the IVIAs according to the first and third embodiments of the invention. As illustrated in FIG. 9(b), the isothermal contour with 43 degrees centigrade of the third embodiment of the invention is shorter in terms of the Z direction and longer in terms of the ρ direction than that of the third embodiment of the invention.

FIG. 9(c) illustrates the temperature distribution patterns of IVIAs according to the second and third embodiments of the invention. As illustrated in FIG. 9(c), the isothermal contour with 43 degrees centigrade of the third embodiment of the invention is shorter in terms of the Z direction and longer in terms of the ρ direction than that of the second embodiment of the invention.

The compared results show that the third embodiment of the invention has the best performance in terms of the thermal distribution pattern, even though the first embodiment of the invention has the best matching performance. Due to the first capacitor together with the second and third capacitors, the IVIAs can be designed with perfect matching and desirable temperature distributions.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. An in-vivo interstitial antenna for thermal treatment and deactivation of tumors including cancers in a human body by means of microwaves comprising:
a coaxial cable having a first conductor, a first insulator surrounding the first conductor, and a second conductor surrounding the first insulator, wherein the first conductor extends from the coaxial cable;
a first capacitor having a second insulator surrounding an end portion of the extension of the first conductor and a third conductor surrounding the second insulator, wherein one end of the third conductor is closed and connected to the first conductor and the other end of the third conductor is open; and
a catheter in which the coaxial cable and the first capacitor are inserted,
wherein the first conductor is a central axis of the coaxial cable and the first capacitor.

2. The in-vivo interstitial antenna according to claim 1, wherein the second conductor in the form of a metal tube surrounds the first conductor concentrically, and the third conductor in the form of a tube surrounds the end portion of the extension of the first conductor concentrically.

3. The in-vivo interstitial antenna according to claim 1, wherein the gap between the coaxial cable, the first capacitor and the catheter comprises air.

4. The in-vivo interstitial antenna according to claim 1, wherein dielectric constants of the first and the second insulators are the same.

5. The in-vivo interstitial antenna according to claim 1, wherein a length of the extension of the first conductor is less than a quarter wavelength.

6. The in-vivo interstitial antenna according to claim 1, wherein the closed end of the first capacitor is flat or convex.

7. The in-vivo interstitial antenna according to claim 6, wherein a cross sectional area of the closed end of the first capacitor is larger than a cross sectional area of the first conductor.

8. An in-vivo interstitial antenna for the thermal treatment and deactivation of tumors including cancers in a human body by means of microwaves comprising:
a coaxial cable having a first conductor, a first insulator surrounding the first conductor, and a second conductor surrounding the first insulator, wherein the first conductor extends from the coaxial cable;
a first capacitor having a second insulator surrounding an end portion of the extension of the first conductor and a third conductor surrounding the second insulator, wherein one end of the third conductor is closed and connected to the first conductor and the other end of the third conductor is open;
a second capacitor having a third insulator surrounding a portion of the extension of the first conductor and a fourth conductor surrounding the third insulator, wherein the second capacitor is formed between the first capacitor and the coaxial cable, and both ends of the fourth conductor are open; and
a catheter in which the coaxial cable, the first capacitor, and the second capacitor are inserted,
wherein the first conductor is a central axis of the coaxial cable, the first and second capacitors.

9. The in-vivo interstitial antenna according to claim 8, wherein the second conductor in the form of a tube surrounds the first conductor concentrically, and the third conductor in the form of a tube surrounds the end portion of the extension of the first conductor concentrically, and the fourth conductor in the form of a tube surrounds the portion of the extension of the first conductor concentrically.

10. The in-vivo interstitial antenna according to claim 8, wherein a space between the coaxial cable, the first capacitor, the second capacitor, and the catheter comprises air.

11. The in-vivo interstitial antenna according to claim 8, wherein a distance between the first capacitor and the second capacitor, a distance between the second capacitor and the coaxial cable, and a length of the second capacitor are the same.

12. The in-vivo interstitial antenna according to claim 8, wherein dielectric constants of the first, the second and the third insulators are the same.

13. The in-vivo interstitial antenna according to claim 8, wherein a length of the extension of the first conductor is less than a quarter wavelength of the microwaves.

14. The in-vivo interstitial antenna according to claim 6, wherein the closed end of the first capacitor is flat or convex.

15. The in-vivo interstitial antenna according to claim 14, wherein a cross sectional area of the closed end of the first capacitor is larger than a cross sectional area of the first conductor.

16. An in-vivo interstitial antenna for thermal treatment and deactivation of tumors including cancers in a human body by means of microwaves comprising:
a coaxial cable having a first conductor, a first insulator surrounding the first conductor, and a second conductor surrounding the first insulator, wherein the first conductor extends from the coaxial cable;
a first capacitor having a second insulator surrounding an end portion of the extension of the first conductor and a third conductor surrounding the second insulator, wherein one end of the third conductor is closed and connected to the first conductor and the other end of the third conductor is open;
a second capacitor having a third insulator surrounding a portion of the extension of the first conductor and a fourth conductor surrounding the third insulator, wherein the second capacitor is formed between the first capacitor and the coaxial cable and both ends of the fourth conductor are open;
a third capacitor having a fourth insulator surrounding a portion of the extension of the first conductor and a fifth conductor surrounding the forth insulator, wherein the third capacitor is formed between the second capacitor and the coaxial cable and both ends of the fifth conductor are open; and
a catheter in which the coaxial cable, the first capacitor, the second capacitor, and the third capacitor are inserted,
wherein the first conductor is a central axis of the coaxial cable, the first, the second, and the third capacitor.

17. The in-vivo interstitial antenna according to claim 16, wherein the second conductor in the form of a tube surrounds the first conductor concentrically, and the third conductor in the form of a tube surrounds the first conductor concentrically, and the fourth conductor in the form of a tube surrounds the first conductor concentrically, and the fifth conductor in the form of a tube surrounds the first conductor concentrically.

18. The in-vivo interstitial antenna according to claim 16, wherein a space between the coaxial cable, the first capacitor, the second capacitor, the third capacitor and the catheter comprises air.

19. The in-vivo interstitial antenna according to claim 16, wherein a distance between the first and the second capacitors, a distance between the second capacitor and the third capacitor, a distance between the third capacitor and the coaxial cable, and a length of the second and the third capacitors are the same.

20. The in-vivo interstitial antenna according to claim 16, wherein dielectric constants of the first, the second, the third and the fourth insulators are the same.

21. The in-vivo interstitial antenna according to claim 16, wherein a length of the extension of the first conductor is less than a quarter wavelength of the microwaves.

22. The in-vivo interstitial antenna according to claim 16, wherein the closed end of the first capacitor is flat or convex.

23. The in-vivo interstitial antenna according to claim 22, wherein a cross sectional area of the closed end of the first capacitor is larger than a cross sectional area of the first conductor.
